(19) United States
(12) Patent Application Publication
(10) Pub. No.: US 2025/0034118 A1
(43) Pub. Date: Jan. 30, 2025

(54) ANTIMALARIAL HEXAHYDROPYRIMIDINE DERIVATIVES

(71) Applicant: UCB BIOPHARMA SRL, Brussels (BE)

(72) Inventors: Alvaro Jesus CARDENAS ARMESTO, Brussels (BE); Teresa DE HARO GARCIA, Brussels (BE); Martin Alexander LOWE, Slough Berkshire (GB)

(21) Appl. No.: 18/705,702

(22) PCT Filed: Oct. 31, 2022
(86) PCT No.: PCT/EP2022/080372

§ 371 Date: Apr. 29, 2024

(30) Foreign Application Priority Data
Nov. 3, 2021 (GB) 2115770.6

(51) Int. Cl. C07D 403/12; A61K 31/513; A61P 33/06

(52) U.S. Cl. CPC C07D 403/12; A61K 31/513; A61P 33/06

(57) ABSTRACT

The compounds of formula (I) as defined herein, being potent inhibitors of the growth and propagation of the Plasmodium falciparum parasite in human blood, are beneficial as pharmaceutical agents, especially in the treatment of malaria.

The present invention relates to a class of heterocyclic compounds, and to their use in therapy. More particularly, this invention is concerned with pharmacologically active substituted hexahydropyrimidine derivatives. These compounds are potent inhibitors of the growth and propagation of the Plasmodium falciparum parasite in human blood, and are accordingly of benefit as pharmaceutical agents, especially in the treatment of malaria.

Malaria is a mosquito-borne infectious disease, caused by a parasite of the genus Plasmodium, which has devastating consequences. Based on 2019 data, approximately 229 million cases, resulting in approximately 409,000 deaths, are reported annually. Approximately 80% of cases occur in sub-Saharan Africa, mostly in young children (aged 5 years or less).

The compounds in accordance with the present invention, being potent inhibitors of the growth and propagation of the P. falciparum parasite in human blood, are therefore beneficial in the treatment of malaria. The compounds of this invention also possess particularly valuable pharmacokinetic and pharmacodynamic properties, and a benign toxicity profile, all of which enhance their attractiveness as therapeutic agents.

WO 2021/032687 describes a particular class of heterocyclic compounds which are stated to be potent inhibitors of the growth and propagation of the P. falciparum parasite in human blood, and therefore to be beneficial in the treatment of malaria.

The present invention provides a compound of formula (I) or a pharmaceutically acceptable salt thereof:

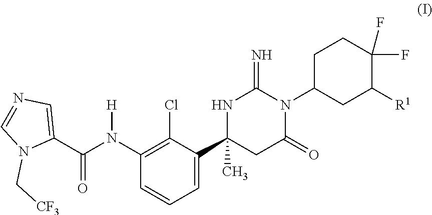

wherein
R¹ represents methyl or methoxy.

The compounds in accordance with the present invention are encompassed within the generic scope of WO 2021/032687. There is, however, no specific disclosure in that publication of a compound of formula (I) as defined above, or a pharmaceutically acceptable salt thereof.

The present invention also provides a compound of formula (I) as defined above, or a pharmaceutically acceptable salt thereof, for use in therapy.

The present invention also provides a compound of formula (I) as defined above, or a pharmaceutically acceptable salt thereof, for use in the treatment and/or prevention of malaria.

The present invention also provides a method for the treatment and/or prevention of malaria which comprises administering to a patient in need of such treatment an effective amount of a compound of formula (I) as defined above, or a pharmaceutically acceptable salt thereof.

The present invention also provides the use of a compound of formula (I) as defined above, or a pharmaceutically acceptable salt thereof, for the manufacture of a medicament for the treatment and/or prevention of malaria.

For use in medicine, the salts of the compounds of formula (I) will be pharmaceutically acceptable salts. Other salts may, however, be useful in the preparation of the compounds of use in the invention or of their pharmaceutically acceptable salts. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002.

The absolute stereochemical configuration of the chiral carbon atom in the imino-tetrahydropyrimidinone nucleus of the compounds according to the invention is as depicted in formula (I) above. Generally, the compounds in accordance with the invention are at least 51% enantiomerically pure (by which it is meant that a sample thereof comprises a mixture of enantiomers containing 51% or more of the enantiomer depicted in formula (I) and 49% or less of the opposite antipode). Typically, the compounds in accordance with the invention are at least 60% enantiomerically pure. Appositely, the compounds in accordance with the invention are at least 75% enantiomerically pure. Suitably, the compounds in accordance with the invention are at least 80% enantiomerically pure. More suitably, the compounds in accordance with the invention are at least 85% enantiomerically pure. Still more suitably, the compounds in accordance with the invention are at least 90% enantiomerically pure. Even more suitably, the compounds in accordance with the invention are at least 95% enantiomerically pure. Preferably, the compounds in accordance with the invention are at least 99% enantiomerically pure. Ideally, the compounds in accordance with the invention are at least 99.9% enantiomerically pure.

Because the compounds of formula (I) have two additional asymmetric centres, they may accordingly exist as enantiomers. Because the compounds in accordance with the invention possess two additional asymmetric centres, they may also exist as diastereomers. The invention is to be understood to extend to the use of all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of formula (I) may exist as tautomers, for example keto (CH₂C═O)↔enol (CH═CHOH) tautomers or amide (NHC═O)↔hydroxyimine (N═COH) tautomers or imide (NHC═NH)↔aminoimine (N═CNH₂) tautomers. Formula (I) and the formulae depicted hereinafter are intended to represent all individual tautomers and all possible mixtures thereof, unless stated or shown otherwise. In addition, the compounds of formula (I) may exist as atropisomers. Formula (I) and the formulae depicted hereinafter are intended to represent all individual atropisomers and all possible mixtures thereof, unless stated or shown otherwise.

Preferably, the R¹-substituted 4,4-difluorocyclohex-1-yl moiety present in the compounds of formula (I) as depicted above is in the 1R,3R or 1S,3S configuration. In a first embodiment, the R¹-substituted 4,4-difluorocyclohex-1-yl moiety is in the 1R,3R configuration. In a second embodiment, the R¹-substituted 4,4-difluorocyclohex-1-yl moiety is in the 1S,3S configuration.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium; D) or ³H (tritium; T) atom, preferably ¹H. Similarly, by way of example, each individual carbon atom present in formula (I), or in the formulae depicted hereinafter, may be present as a ¹²C, ¹³C or ¹⁴C atom, preferably ¹²C.

In a first embodiment, R¹ represents methyl. In a second embodiment, R¹ represents methoxy.

In a first aspect, the present invention provides the compound of formula (IA):

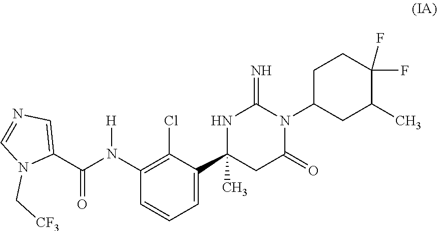 [SMILES: CC1CC(N2C(=N)N[C@](C)(c3cccc(NC(=O)c4cncn4CC(F)(F)F)c3Cl)CC2=O)CCC1(F)F]

or a pharmaceutically acceptable salt thereof.

Preferably, the 4,4-difluoro-3-methylcyclohex-1-yl moiety present in the compound of formula (IA) as depicted above is in the 1R,3R or 1S,3S configuration. In a particular embodiment, the 4,4-difluoro-3-methylcyclohex-1-yl moiety is in the 1R,3R configuration. In another embodiment, the 4,4-difluoro-3-methylcyclohex-1-yl moiety is in the 1S,3S configuration.

In a second aspect, the present invention provides the compound of formula (IB):

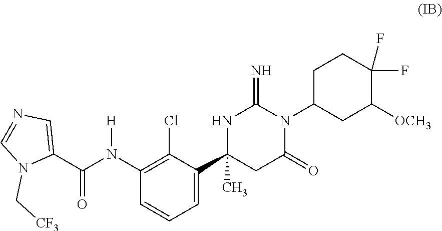 [SMILES: COC1CC(N2C(=N)N[C@](C)(c3cccc(NC(=O)c4cncn4CC(F)(F)F)c3Cl)CC2=O)CCC1(F)F]

or a pharmaceutically acceptable salt thereof.

Preferably, the 4,4-difluoro-3-methoxycyclohex-1-yl moiety present in the compound of formula (IB) as depicted above is in the 1R,3R or 1S,3S configuration. In a first embodiment, the 4,4-difluoro-3-methylcyclohex-1-yl moiety is in the 1R,3R configuration. In a second embodiment, the 4,4-difluoro-3-methylcyclohex-1-yl moiety is in the 1S,3S configuration.

Specific novel compounds in accordance with the present invention include each of the compounds whose preparation is described in the accompanying Examples, and pharmaceutically acceptable salts thereof.

The present invention also provides a pharmaceutical composition which comprises a compound in accordance with the invention as described above, or a pharmaceutically acceptable salt thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds of formula (I) may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds of formula (I) may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds in accordance with the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds in accordance with the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds in accordance with the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration compounds may be formulated in an ointment such as petrolatum.

For rectal administration the compounds in accordance with the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound in accordance with the invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

General methods for the preparation of the compounds of formula (I) as defined above are described in WO 2021/032687.

The compounds in accordance with the invention may be prepared by a process which comprises the following steps:
(i) treating 3-(2,2,2-trifluoroethyl) imidazole-4-carboxylic acid with oxalyl chloride and N,N-dimethylformamide; and
(ii) reacting the acid chloride derivative thereby obtained with a compound of formula (III):

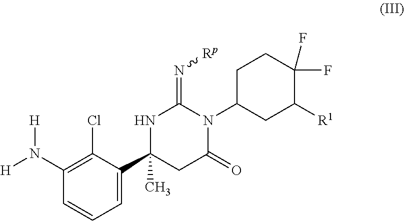

wherein R¹ is as defined above, and R^{p} represents hydrogen or an N-protecting group; followed, as necessary, by removal of the N-protecting group R^{p}.

Step (i) is conveniently accomplished at ambient temperature in a suitable solvent, e.g. a chlorinated solvent such as dichloromethane.

Step (ii) is conveniently carried out in the presence of a base, e.g. an organic base such as triethylamine, or pyridine. The reaction is typically performed at 0° C. to ambient temperature in a suitable solvent, e.g. a chlorinated solvent such as dichloromethane.

Suitably, the N-protecting group Rₚ is tert-butoxycarbonyl (BOC).

Where the N-protecting group Rₚ is BOC, subsequent removal of the BOC group may suitably be accomplished by treatment with an acid, e.g. a mineral acid such as hydrochloric acid, or an organic acid such as trifluoroacetic acid. The reaction will typically be effected at ambient temperature in a suitable solvent, e.g. a chlorinated solvent such as dichloromethane, or a cyclic ether such as 1,4-dioxane.

In an alternative procedure, the compounds in accordance with the invention may be prepared by a process which comprises reacting 3-(2,2,2-trifluoroethyl) imidazole-4-carboxylic acid with a compound of formula (III) as defined above; in the presence of a coupling agent; followed, as necessary, by removal of the N-protecting group Rₚ.

Suitably, the coupling agent may be chloro-N,N,N′,N′-tetramethylformamidinium hexafluorophosphate, in which case the reaction may generally be carried out in the presence of 1-methylimidazole. The reaction is conveniently performed at ambient temperature in a suitable solvent, e.g. a nitrile solvent such as acetonitrile.

Alternatively, the coupling agent may be 2,4,6-tripropyl-1,3,5,2,4,6-trioxa-triphosphorinane 2,4,6-trioxide, in which case the reaction may generally be carried out in the presence of a base which may suitably include organic amines, e.g. a trialkylamine such as N,N-diisopropylethylamine, or an aromatic base such as pyridine. The reaction is conveniently performed at ambient or elevated temperature in a suitable solvent, e.g. a chlorinated solvent such as dichloromethane, or an organic ester solvent such as ethyl acetate.

Alternatively, the coupling agent may be 2-chloro-1-methylpyridinium iodide, in which case the reaction may generally be carried out in the presence of a base, e.g. a trialkylamine such as N,N-diisopropylethylamine. The reaction is conveniently performed at ambient or elevated temperature in a suitable solvent, e.g. a chlorinated solvent such as dichloromethane, or an organic nitrile solvent such as acetonitrile.

3-(2,2,2-Trifluoroethyl) imidazole-4-carboxylic acid may suitably be prepared by a method analogous to the procedure described in Journal of Fluorine Chemistry, 2000, 106, 189-197.

The intermediates of formula (III) above may be prepared by treating a compound of formula (IV):

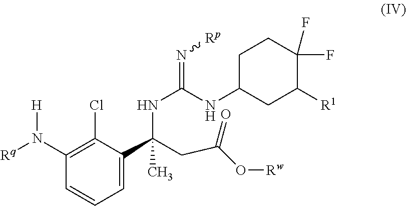

wherein R¹ and R^{p} are as defined above, R^{q} represents an N-protecting group, and R^{w} represents C₁₋₄ alkyl, especially methyl; with a base; followed by removal of the N-protecting group R^{q}.

Suitably, the base of use in the above reaction is a C₁₋₄ alkoxide salt, typically an alkali metal alkoxide such as potassium tert-butoxide. The reaction is conveniently accomplished at 0° C. to ambient temperature in a suitable solvent, e.g. a cyclic ether such as tetrahydrofuran.

Suitably, the N-protecting group R^{q} is benzyloxycarbonyl.

Where the N-protecting group R^{q} is benzyloxycarbonyl, subsequent removal of the benzyloxycarbonyl group may suitably be accomplished by catalytic hydrogenation. Typically, this will involve treatment with gaseous hydrogen in the presence of a hydrogenation catalyst such as palladium on charcoal.

The intermediates of formula (IV) above may be prepared by reacting a compound of formula (V) with a compound of formula (VI):

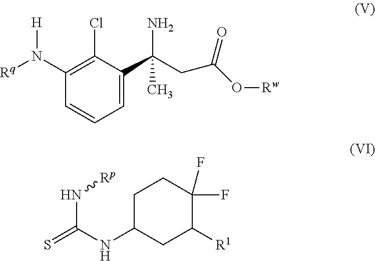

wherein R¹, R^{p}, R^{q} and R^{w} are as defined above.

Generally, the reaction between compounds (V) and (VI) is performed in the presence of a coupling agent. A suitable coupling agent is N-(3-dimethylaminopropyl)-N′-ethylcarbodiimide (EDCI). Suitably, the reaction is performed in the presence of a base, typically an organic base such as N,N-diisopropylethylamine.

The reaction between compounds (V) and (VI) is conveniently accomplished at ambient temperature in a suitable solvent, e.g. a dipolar aprotic solvent such as N,N-dimethylformamide.

Where they are not commercially available, the starting materials of formula (V) and (VI) may be prepared by methods analogous to those described in the accompanying Examples, or by standard methods well known from the art.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt. In another resolution process a racemate of formula (I) may be separated using chiral HPLC. Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode. Chromatography, recrystallisation and other conventional separation procedures may also be used with intermediates or final products where it is desired to obtain a particular geometric isomer of the invention.

During any of the above synthetic sequences it may be necessary and/or desirable to protect sensitive or reactive groups on any of the molecules concerned. This may be achieved by means of conventional protecting groups, such as those described in Greene's Protective Groups in Organic Synthesis, ed. P. G. M. Wuts, John Wiley & Sons, 5^{th} edition, 2014. The protecting groups may be removed at any convenient subsequent stage utilising methods known from the art.

The following Examples illustrate the preparation of compounds according to the invention.

The compounds of the present invention are potent inhibitors of the growth and propagation of the Plasmodium falciparum parasite in human blood. As such, they are active in a P. falciparum 3D7 asexual blood stage assay, generally exhibiting IC₅₀ values of 5 nM or better (the skilled person will appreciate that a lower IC₅₀ figure denotes a more active compound).

### Asexual Blood Stage Assay

The assay used to measure the effect of test compounds on a bloodstream stage of Plasmodium falciparum 3D7 strain employs SYBR green as the readout. This is a dye that binds to double stranded deoxyribonucleic acid (DNA) with a resulting increase in fluorescence, allowing detection of P. falciparum DNA in infected erythrocytes, and thereby providing a measure of parasite growth and propagation.

### P. Falciparum Culture Maintenance

Erythrocytes (A+ blood) were prepared for both parasite culture and assay by washing 4 times with incomplete media (15.9 g RPMI 1640 (25 mM HEPES, L-glutamine), 1 g NaHCO₃, 2 g glucose, 400 μL gentacin (500 mg/mL), 2 mL hypoxanthine solution (13.6 g/L in 0.1M NaOH pH 7.3) in 1 litre of media). The cells were centrifuged at 1800 g for 5 minutes, before decanting the supernatant and re-suspending in fresh incomplete media. On the final wash, the cells were re-suspended in complete media (incomplete media with 5 g/L AlbumaxII), and centrifuged at 1800 g for 3 minutes. This cell sediment was treated as 100% haematocrit.

P. falciparum 3D7 was cultured in erythrocytes at 5% haematocrit in complete media at 37° C. (1% O₂, 3% CO₂, balance N₂). Cultures were split on a weekly basis to achieve a 1% parasitaemia in erythrocites at 5% haematocrit in fresh media. Culture media is replaced by fresh media every other day (2 times during the week).

### Assay Procedure

On day 1, test compounds were added to assay plates using Echo dispensing technology (1.5 fold dilution and 20 points titration). 50 nL of each compound dilution was added to 50 μL of culture (5% haematocrit, 0.5% parasitaemia) and incubated for 72 h at 37° C. (1% O₂, 3% CO₂, balance N₂). Final concentrations of test compounds ranged from 50,000 nM to 15 nM, in 0.5% DMSO.

On day 4, 10 μL SYBR green (Invitrogen S7563 supplied as 10,000× concentrate in DMSO) pre-diluted to 3× concentrate with Lysis buffer (20 mM Tris pH 7.9, 5% EDTA, 0.16% w/v, 1.6% TX100 v/v) was added to the cultures and incubated in the dark, overnight, at room temperature.

On day 5, fluorescent signal was measured using a BioTek plate reader (excitation 485 nm, emission 528 nm). All data were processed using IDBS ActivityBase. Raw data were converted into per cent inhibition through linear regression by setting the high inhibition control (mefloquine) as 100% and the no inhibition control (DMSO) as 0%.

Quality control criteria for passing plates were as follows: Z′>0.5, S:B>3, % CV_{(no inhibition control)}<15. The formula used to calculate Z′ is:

$$1 - \frac{3 ( σ p + σ n )}{( μ p - μ n )}$$

where μ denotes the mean; σ denotes the standard deviation; p denotes the positive control; and n denotes the negative control.

All EC₅₀ curve fitting was undertaken using the following bi-phasic two site dose response using XLfit model 300 (IDBS):

$$y - \frac{A}{1 + {10}^{( C - log 10 ( Bx ) )}} + \frac{100 - A}{1 + {10}^{( D - log 10 ( Bx ) )}}$$

where A=100 minus the top of the upper curve 1 and the bottom of lower curve; B=Hill slope; log(C)=IC₅₀ concentration at lower site; log(D)=IC₅₀ concentration at upper site; x=inhibitor concentration; and y=% inhibition.

When tested in the P. falciparum 3D7 asexual blood stage assay as described above, the compounds of the accompanying Examples were found to exhibit the following IC₅₀ values.

| Example | IC₅₀ (nM) |
|---|---|
| 1 | 5.0 |
| 2 | 4.2 |

### EXAMPLES

Abbreviations

| | |
|---|---|
| DCM: dichloromethane | EtOAc: ethyl acetate |
| DMSO: dimethyl sulfoxide | THF: tetrahydrofuran |
| MeOH: methanol | DMF: N,N-dimethylformamide |
| DIPEA: N,N-diisopropylethylamine | TFA: trifluoroacetic acid |
| TFAA: trifluoroacetic anhydride | EtOH: ethanol |
| DAST: diethylaminosulfur trifluoride | DEA: diethylamine |
| TBAF: tetra-n-butylammonium fluoride | M: mass |
| EDCI: N-(3-dimethylaminopropyl)- | RT: retention time |
| N′-ethylcarbodiimide | |
| T3P ®: 2,4,6-tripropyl-1,3,5,2,4,6- | |
| trioxatriphosphorinane- | |
| 2,4,6-trioxide solution | |
| h: hour | |
| r.t .: room temperature | |
| DAD: Diode Array Detector | |
| HPLC: High Performance Liquid | |
| Chromatography | |
| LCMS: Liquid Chromatography | |
| Mass Spectrometry | |
| ESI: Electrospray Ionisation | |
| VCD: Vibrational Circular Dichroism | |

### Nomenclature

Compounds were named in accordance with IUPAC guidelines with the aid of Biovia Draw version 19.1.

The asterisk (*)—for example, in compounds designated (1S*,3S*)—indicates compounds of known relative stereochemistry but unknown absolute stereochemistry.

### Analytical Conditions

### Method 1

Column: Waters X Bridge C18, 2.1×30 mm, 2.5 μm
Injection Volume 5.0 μL
Flow Rate 1.00 mL/minute
Detection:
   MS—ESI+ m/z 150 to 800
   UV—DAD 220-400 nm
Solvent A: 5 mM ammonium formate in water+0.1% ammonia
Solvent B: acetonitrile+5% Solvent A+0.1% ammonia
Gradient program:
   5% B to 95% B in 4.0 minutes; hold until 5.00 minutes;
   at 5.10 minutes concentration of B is 5%; hold up to 6.5 minutes

### Method 2

Column: Waters UPLC X Bridge BEH (C18, 2.1×50 mm, 2.5 μm)
Temperature: 45° C.
Injection volume: 1.0 μL
Flow rate: 1.00 mL/minute
Detection: Mass spectrometry—+/− detection in the same run
PDA: 210 to 400 nm
Solvent A: 10 mM ammonium formate in water+0.1% formic acid
Solvent B: 95% acetonitrile+5% H₂O+0.1% formic acid

| Time | % A | % B |
|---|---|---|
| 0 | 95 | 5 |
| 0.10 | 95 | 5 |
| 2.10 | 5 | 95 |
| 2.35 | 5 | 95 |
| 2.80 | 95 | 5 |

### INTERMEDIATE 1

### (8-Oxo-1,4-dioxaspiro[4.5]decan-7-yl) acetate

A mixture of 1,4-dioxaspiro[4.5]decan-8-one (7.50 g, 48.0 mmol) and lead (IV) acetate (31.9 g, 72.0 mmol) in toluene (150 mL) was heated at 110° C. for 16 h, after which time the reaction mixture was cooled to r.t., filtered through a pad of Celite® and washed with EtOAc (3×150 mL). The organic layer was separated, then dried over anhydrous Na₂SO₄ and concentrated in vacuo. The crude residue was purified by column chromatography (silica, 100-200 mesh, 20% EtOAc in hexanes) to afford the title compound (6.56 g, 64%) as an off-white solid. δ_{H} (400 MHz, DMSO-d₆) 5.24 (dd, J 13.2, 6.8 Hz, 1H), 3.89-4.08 (m, 4H), 2.54-2.69 (m, 1H), 2.23-2.33 (m, 2H), 2.07 (s, 3H), 1.93-2.01 (m, 2H). One proton signal merged with solvent peak.

### INTERMEDIATE 2

### (8,8-Difluoro-1,4-dioxaspiro[4.5]decan-7-yl) acetate

To a solution of Intermediate 1 (13.5 g, 63.0 mmol) in dry DCM (270 mL) was added DAST (20.8 mL, 158 mmol) at 0° C. The reaction mixture was stirred at 0° C. for 30 minutes, then heated at 50° C. for 5 h, after which time the reaction mixture was quenched with saturated aqueous NaHCO₃ solution (450 mL) and extracted with DCM (3×400 mL). The organic layer was separated, then dried over anhydrous Na₂SO₄ and concentrated in vacuo. The crude residue was purified by column chromatography (silica, 100-200 mesh, 15% EtOAc in hexanes) to afford the title compound (9.51 g, 64%) as a colourless oil. δ_{H} (400 MHz, DMSO-d₆) 5.18-5.30 (m, 1H), 3.85-3.99 (m, 4H), 2.10-2.25 (m, 1H), 2.08 (s, 3H), 1.98-2.06 (m, 2H), 1.87-1.98 (m, 1H), 1.72-1.81 (m, 2H).

### INTERMEDIATE 3

### 4,4-Difluoro-3-hydroxycyclohexanone

To a solution of Intermediate 2 (9.50 g, 40.2 mmol) in THF (95 mL) was added 10% aqueous HCl (380 mL) slowly at r.t. The reaction mixture was stirred at r.t. for 16 h, then quenched with saturated aqueous NaHCO₃ solution (450 mL) and extracted with DCM (3×450 mL). The organic layer was separated, washed with saturated aqueous NH₄Cl solution (200 mL) and brine (200 mL), then dried over anhydrous Na₂SO₄ and concentrated in vacuo, to afford the crude title compound (4.50 g) as a colourless oil, which was utilised without further purification. δ_{H} (400 MHz, CDCl₃) 4.22-4.33 (m, 1H), 3.74 (br s, 1H), 2.77-2.88 (m, 1H), 2.45-2.68 (m, 4H), 2.18-2.32 (m, 1H).

### INTERMEDIATE 4

### 3-[tert-Butyl(dimethyl)silyl]oxy-4,4-difluorocyclohexanone

To a solution of Intermediate 3 (4.50 g, 30.0 mmol) in dry DCM (90 mL) was added imidazole (5.10 g, 74.9 mmol). The reaction mixture was cooled to 0° C., then tert-butyldimethylsilyl chloride (11.3 g, 74.9 mmol) was added. The reaction mixture was stirred at r.t. for 16 h, then quenched with 10% aqueous NH₄Cl solution (150 mL) and extracted with EtOAc (3×150 mL). The organic layer was separated, then dried over anhydrous Na₂SO₄ and concentrated in vacuo. The crude residue was purified by column chromatography (silica, 100-200 mesh, 30% EtOAc in hexanes) to afford the title compound (7.00 g, 88%) as a colourless oil. δ_{H} (400 MHz, CDCl₃) 4.18-4.27 (m, 1H), 2.80 (d, J 14.6 Hz, 1H), 2.34-2.60 (m, 4H), 2.08-2.28 (m, 1H), 0.89 (s, 9H), 0.11 (s, 6H).

### INTERMEDIATE 5

### rac-(1R,3R)-N-Benzyl-3-[tert-butyl(dimethyl) silyl]oxy-4,4-difluorocyclohexanamine

To a solution of Intermediate 4 (7.00 g, 26.5 mmol) in isopropanol (70 mL) were added benzylamine (4.34 mL, 39.7 mmol) and acetic acid (0.50 mL) under a nitrogen atmosphere. The reaction mixture was stirred at r.t. for 8 h, then NaBH₄ (2.50 g, 39.7 mmol) was added. The reaction mixture was stirred at r.t. for 16 h, then quenched with cold water (500 mL) and extracted with EtOAc (3×150 mL). The organic layer was separated, then dried over anhydrous Na₂SO₄ and concentrated in vacuo. The crude residue was purified by column chromatography (silica, 100-200 mesh, 30% EtOAc in hexanes) to afford the title compound (8.00 g, 71%) as a pale yellow oil. δ_{H} (400 MHz, CDCl₃) 7.32-7.44 (m, 5H), 3.87 (s, 2H), 3.74-3.83 (m, 1H), 2.76-2.86 (m, 2H), 2.08-2.25 (m, 2H), 1.98-2.07 (m, 1H), 1.61-1.96 (m, 3H), 0.88 (s, 9H), 0.09 (s, 6H). LCMS (Method 1, ESI): 356.10 [MH]⁺, RT 2.57 minutes.

### INTERMEDIATE 6

### rac-(1R,5R)-5-(Benzylamino)-2,2-difluorocyclohexanol

To a solution of Intermediate 5 (4.00 g, 9.39 mmol) in THF (60 mL) was added TBAF (1M in THF, 14.1 mL) under a nitrogen atmosphere at 0° C. The reaction mixture was stirred at r.t. for 2 h, then quenched with water (200 mL) and extracted with EtOAc (3×300 mL). The organic layer was separated, then dried over anhydrous Na₂SO₄ and concentrated in vacuo. The crude residue was purified by combi-flash chromatography (40% EtOAc in hexanes) to afford the title compound (1.90 g, 84%) as a colourless oil. δ_{H} (400 MHz, DMSO-d₆) 1.22-1.44 (m, 2H), 1.59-1.85 (m, 2H), 1.88-2.07 (m, 2H), 7.30 (q, J 7.66 Hz, 4H), 7.13-7.24 (m, 1H), 5.48-5.62 (m, 1H), 3.69 (s, 2H), 3.53-3.66 (m, 1H), 2.53-2.66 (m, 1H). One exchangeable proton signal not observed in NMR spectrum.

### INTERMEDIATE 7

### tert-Butyl N-benzyl-N-[rac-(1R,3R)-4,4-difluoro-3-hydroxycyclohexyl]carbamate

To a solution of Intermediate 6 (1.90 g, 7.87 mmol) in DCM (20 mL) was added triethylamine (2.20 mL, 15.7 mmol). The reaction mixture was stirred at r.t. for 15 minutes, then di-tert-butyl dicarbonate (2.06 g, 9.45 mmol) was added at 0° C. The reaction mixture was stirred at r.t. for 16 h, then diluted with DCM (150 mL) and washed with water (100 mL). The organic layer was separated, then dried over anhydrous Na₂SO₄ and concentrated in vacuo. The crude residue was purified by combi-flash chromatography (20% EtOAc in hexanes) to afford the title compound (1.80 g, 67%) as an off-white solid. δ_{H} (400 MHz, DMSO-d₆) 7.28-7.37 (m, 2H), 7.17-7.27 (m, 3H), 5.37 (d, J 5.38 Hz, 1H), 4.34 (s, 2H), 3.92 4.07 (m, 1H), 3.55-3.75 (m, 1H), 1.89-2.02 (m, 1H), 1.40-1.84 (m, 5H), 1.34 (s, 9H). LCMS (Method 1, ESI): 286.00 [MH]⁺, RT 2.11 minutes.

### INTERMEDIATE 8

### tert-Butyl N-benzyl-N-[rac-(1R,3R)-4,4-difluoro-3-methoxycyclohexyl]carbamate

To a solution of Intermediate 7 (1.50 g, 4.38 mmol) in THF (30 mL) was added NaH (0.26 g, 6.57 mmol) at 0° C. under a nitrogen atmosphere. The reaction mixture was stirred at r.t. for 30 minutes, then iodomethane (0.93 g, 6.57 mmol) was added. The reaction mixture was stirred at r.t. for 2 h, then poured into water (160 mL) and extracted with EtOAc (2×160 mL). The organic layer was separated, then dried over anhydrous Na₂SO₄ and concentrated in vacuo, to afford the crude title compound (1.72 g) as a pale yellow semi-solid, which was utilised without further purification. δ_{H} (400 MHz, CDCl₃) 7.13-7.37 (m, 5H), 4.20-4.50 (m, 3H), 3.50 (s, 3H), 3.25-3.45 (m, 1H), 2.02-2.19 (m, 2H), 1.64-1.80 (m, 3H), 1.40 (s, 9H), 0.80-0.96 (m, 1H).

### INTERMEDIATE 9

### rac-(1R,3R)-N-Benzyl-4,4-difluoro-3-methoxycyclohexanamine

To a solution of Intermediate 8 (1.70 g, 4.78 mmol) in DCM (20 mL) was added TFA (1.10 mL, 14.3 mmol) at 0° C. The reaction mixture was stirred at r.t. for 2 h, then poured into cold saturated aqueous NaHCO₃ solution (80 mL) and extracted with EtOAc (3×80 mL). The organic layer was separated, then dried over anhydrous Na₂SO₄ and concentrated in vacuo. The crude residue was purified by combi-flash chromatography (40% EtOAc in hexanes) to afford the title compound (1.10 g, 90%) as a colourless oil. δ_{H} (400 MHz, DMSO-d₆) 7.25-7.38 (m, 4H), 7.16-7.24 (m, 1H), 3.71 (s, 2H), 3.40-3.53 (m, 1H), 3.37 (s, 3H), 2.55-2.63 (m, 1H), 2.10-2.26 (m, 2H), 1.92-2.05 (m, 1H), 1.82-1.92 (m, 1H), 1.60-1.80 (m, 1H), 1.10-1.26 (m, 2H).

### INTERMEDIATE 10

### rac-(1R,3R)-4,4-Difluoro-3-methoxy-cyclohexanamine

To a solution of Intermediate 9 (1.10 g, 4.31 mmol) in MeOH (22 mL) was added 20% Pd/C (0.23 g, 0.43 mmol) under a nitrogen atmosphere. The reaction mixture was stirred at r.t. for 2 h under hydrogen pressure, then filtered through a pad of Celite®, washing through with MeOH (3×30 mL). The filtrate was concentrated in vacuo to afford the title compound (0.67 g, 94%) as a colourless oil, which was utilised without further purification. δ_{H} (400 MHz, DMSO-d₆) 3.40-3.53 (m, 1H), 2.73 (t, J 10.76 Hz, 1H), 1.88-2.08 (m, 2H), 1.64-1.85 (m, 2H), 1.07-1.26 (m, 2H). Three proton signals merged into solvent peak; and two exchangeable proton signals not observed in NMR spectrum.

### INTERMEDIATE 11

### tert-Butyl N-{[rac-(1R,3R)-4,4-difluoro-3-methoxycyclohexyl]carbamothioyl}carbamate

To a solution of N,N′-bis-tert-butoxycarbonylthiourea (1.09 g, 3.94 mmol) in THF (8 mL) was added NaH (0.47 g, 11.8 mmol) at 0° C. The reaction mixture was stirred at 0° C. for 1 h, then TFAA (0.83 mL, 5.90 mmol) was added at 0° C. The reaction mixture was stirred at 0° C. for 1 h, then a solution of Intermediate 10 (0.65 g, 3.94 mmol) in THF (2 mL) was added at 0° C. The reaction mixture was stirred at r.t. for 16 h, then diluted with water (70 mL) and extracted with EtOAc (2×70 mL). The organic layer was separated and washed with brine (70 mL), then dried over anhydrous Na₂SO₄ and concentrated in vacuo. The crude residue was purified by column chromatography (silica, 100-200 mesh, 20% EtOAc in hexanes) to afford the title compound (1.10 g, 85%) as a pale yellow solid. δ_{H} (400 MHz, DMSO-d₆) 10.58 (s, 1H), 10.20 (d, J 6.85 Hz, 1H), 4.40-4.52 (m, 1H), 3.59-3.70 (m, 1H), 3.40 (s, 3H), 1.70-2.15 (m, 6H), 1.44 (s, 9H). LCMS (Method 1, ESI): 325.10 [MH]⁺, RT 1.98 minutes.

### INTERMEDIATE 12

### Methyl (3S)-3-[3-(benzyloxycarbonylamino)-2-chlorophenyl]-3-({(Z)-N′-tert-butoxy-carbonyl-N-[rac-(1R,3R)-4,4-difluoro-3-methoxycyclohexyl]carbamimidoyl}amino)-butanoate

To a solution of Intermediate 11 (1.10 g, 3.36 mmol) and methyl (3S)-3-amino-3-[3-(benzyloxycarbonylamino)-2-chlorophenyl]butanoate (cf. WO 2021/032687, Intermediate 5) (1.39 g, 3.36 mmol) in DMF (10 mL) were added EDCI (0.96 g, 5.03 mmol) and DIPEA (1.17 mL, 6.71 mmol) at 0° C. The reaction mixture was stirred at r.t. for 16 h, then quenched with water (140 mL) and extracted with EtOAc (3×70 mL). The organic layer was separated, then dried over anhydrous Na₂SO₄ and concentrated in vacuo, to afford the crude title compound (2.80 g) as a pale yellow solid, which was utilised without further purification. LCMS (Method 1, ESI): 667.10 [MH]⁺, RT 2.35 minutes.

### INTERMEDIATE 13

### tert-Butyl (NE)-N-{(4S)-4-[3-(benzyloxycarbonylamino)-2-chlorophenyl]-1-[(1RS,3RS)-4,4-difluoro-3-methoxycyclohexyl]-4-methyl-6-oxohexahydropyrimidin-2-ylidene}-carbamate

To a solution of Intermediate 12 (2.80 g, 3.08 mmol) in dry THF (28 mL) was added potassium tert-butoxide (1M in THF, 3.08 mL) at 0° C. The reaction mixture was stirred at 0° C. for 1.5 h, then quenched with water (150 mL) and extracted with EtOAc (3×80 mL). The organic layer was separated, then dried over anhydrous Na₂SO₄ and concentrated in vacuo. The residue was purified by combi-flash chromatography (25% EtOAc in hexanes) to afford the title compound (1.88 g, 94%) as an off-white solid. δ_{H} (400 MHz, DMSO-d₆) 10.51 (d, J 2.45 Hz, 1H), 9.24 (s, 1H), 7.58 (d, J 7.82 Hz, 1H), 7.29-7.45 (m, 6H), 7.18 (d, J 4.40 Hz, 1H), 5.14 (s, 2H), 4.45-4.63 (m, 1H), 3.59 (d, J 16.63 Hz, 1H), 3.36-3.53 (m, 1H), 3.26-3.35 (s, 3H), 2.16-2.43 (m, 2H), 1.83-2.05 (m, 2H), 1.76 (s, 3H), 1.56-1.70 (m, 1H), 1.45 (s, 9H), 1.26-1.35 (m, 1H), 0.95-1.07 (m, 1H). LCMS (Method 1, ESI): 635.05 [MH]⁺, RT 2.36 minutes.

### INTERMEDIATE 14

### tert-Butyl (NE)-N-{(4S)-4-(3-amino-2-chlorophenyl)-1-[(1RS,3RS)-4,4-difluoro-3-methoxycyclohexyl]-4-methyl-6-oxohexahydropyrimidin-2-ylidene}carbamate

To a solution of Intermediate 13 (1.88 g, 2.88 mmol) in MeOH (30 mL) was added 20% Pd/C (0.23 g, 0.43 mmol) under an argon atmosphere. The reaction mixture was stirred at r.t. for 45 minutes under hydrogen pressure, then filtered through a pad of Celite®, washing through with MeOH (4×40 mL). The filtrate was dried over anhydrous Na₂SO₄ and concentrated in vacuo. The crude residue was purified by combi-flash chromatography (20% EtOAc in hexanes) to afford the title compound (1.21 g, 79%) as an off-white solid. δ_{H} (400 MHz, DMSO-d₆) 10.46 (s, 1H), 6.95-7.08 (m, 1H), 6.75-6.83 (m, 1H), 6.40-6.50 (m, 1H), 5.52 (s, 2H), 4.49-4.67 (m, 1H), 3.44-3.60 (m, 2H), 3.28-3.36 (s, 3H), 3.05-3.16 (m, 1H), 2.18-2.42 (m, 2H), 1.84-2.07 (m, 1H), 1.74-1.84 (m, 1H), 1.73 (s, 3H), 1.45 (s, 9H), 1.21-1.31 (m, 1H), 0.81-0.91 (m, 1H). LCMS (Method 1, ESI): 500.95 [MH]⁺, RT 2.06 minutes.

### INTERMEDIATES 15 & 16

### tert-Butyl N-{[(1R,3R)-4,4-difluoro-3-methylcyclohexyl]carbamothioyl}carbamate (Intermediate 15)

### tert-Butyl N-{[(1S,3S)-4,4-difluoro-3-methylcyclohexyl]carbamothioyl}carbamate (Intermediate 16)

Prepared from (1R*,3R*)-4,4-difluoro-3-methylcyclohexanamine hydrochloride (cf. WO 2021/032687, Intermediate 32) (2.70 mmol) in accordance with the procedure described for Intermediate 11. The resulting material (1.60 g) was subjected to chiral preparative HPLC (Column: AD; Mobile Phase: EtOH 10%/heptane 90%/DEA 0.1%; Temperature: 30° C.; Wavelength: 240 nm, Flow: 1.5 mL/minute) to afford the title compounds (Example 15, RT 2.94 minutes; and Example 16, RT 3.70 minutes) as off-white solids. δ_{H} (400 MHz, DMSO-d₆) 10.63-10.62 (br s, 1H), 9.69-9.68 (br s, 1H), 4.28-4.26 (m, 1H), 2.06-2.02 (m, 4H), 1.94-1.83 (m, 1H), 1.52-1.69 (m, 1H), 1.43 (s, 9H), 1.34-1.25 (m, 1H), 0.96 (d, 3H).

The absolute stereochemical configuration of the title compounds was confirmed by applying VCD analysis with a confidence level of 98%.

### INTERMEDIATE 17

### tert-Butyl (NE)-N-{(4S)-4-[3-(benzyloxycarbonylamino)-2-chlorophenyl]-1-[(1R,3R)-4,4-difluoro-3-methylcyclohexyl]-4-methyl-6-oxohexahydropyrimidin-2-ylidene}carbamate

Prepared from Intermediate 15 (2.50 g, 2.99 mmol) in accordance with the procedure described for Intermediate 12, followed by the procedure described for Intermediate 13, to afford the title compound (2.40 g, 88%) as an off-white solid. LCMS (Method 1, ESI): 619.40 [MH]⁺, RT 1.68 minutes.

### INTERMEDIATE 18

### tert-Butyl (NE)-N-{(4S)-4-(3-amino-2-chlorophenyl)-1-[(1R,3R)-4,4-difluoro-3-methyl-cyclohexyl]-4-methyl-6-oxohexahydropyrimidin-2-ylidene}carbamate

Prepared from Intermediate 17 (2.40 g, 2.60 mmol) in accordance with the procedure described for Intermediate 14 to afford the title compound (0.84 g, 57%) as an off-white solid. LCMS (Method 1, ESI): 485.25 [MH]⁺, RT 2.35 minutes.

### INTERMEDIATE 19

### 3-(2,2,2-Trifluoroethyl)imidazole-4-carbonyl chloride

To a solution of 3-(2,2,2-trifluoroethyl) imidazole-4-carboxylic acid (Journal of Fluorine Chemistry, 2000, 106, 189-197) (0.13 g, 0.64 mmol) in DCM (5 mL) and DMF (0.02 mL) was added oxalyl chloride (0.09 mL, 1.00 mmol) at 0° C. The reaction mixture was stirred at r.t. for 4 h, then concentrated in vacuo at 25° C., to afford the title compound as an off-white semi-solid, which was utilised without further purification.

### INTERMEDIATE 20

### tert-Butyl (NE)-N-{(4S)-4-(2-chloro-3-{[3-(2,2,2-trifluoroethyl)imidazole-4-carbonyl]-amino}phenyl)-1-[(1R,3R)-4,4-difluoro-3-methylcyclohexyl]-4-methyl-6-oxohexahydro-pyrimidin-2-ylidene}carbamate

To a solution of Intermediate 18 (0.13 g, 0.26 mmol) and Intermediate 19 (0.14 g, 0.65 mmol) in dry DCM (10 mL) was added pyridine (0.06 mL, 0.78 mmol) at 0° C. The reaction mixture was stirred at r.t. for 1 h, then quenched with water (50 mL) and extracted with DCM (2×150 mL). The organic layer was separated and concentrated in vacuo. The crude residue was purified by column chromatography (silica, 100-200 mesh, 50% EtOAc in hexanes) to afford the title compound (0.09 g, 49%) as an off-white solid. δ_{H} (400 MHz, DMSO-d₆) 0.82 (d, J 5.38 Hz, 3H), 0.96-1.09 (m, 1H), 1.21-1.30 (m, 1H), 1.45 (s, 9H), 1.50-1.61 (m, 1H), 1.79 (s, 3H), 2.01-2.24 (m, 3H), 2.87-3.09 (m, 1H), 3.61 (d, J 15.65 Hz, 1H), 3.93-4.11 (m, 1H), 4.45-4.72 (m, 1H), 5.23-5.52 (m, 2H), 7.30 (d, J 6.36 Hz, 1H), 7.36-7.54 (m, 2H), 7.94 (s, 1H), 8.05 (s, 1H), 10.12 (s, 1H), 10.54 (s, 1H). LCMS (Method 1, ESI): 661.15 [MH]⁺, RT 2.27 minutes.

### INTERMEDIATE 21

### tert-Butyl (NE)-N-{(4S)-4-(2-chloro-3-{[3-(2,2,2-trifluoroethyl)imidazole-4-carbonyl]-amino}phenyl)-1-[(1S*, 3S*)-4,4-difluoro-3-methoxycyclohexyl]-4-methyl-6-oxo-hexahydropyrimidin-2-ylidene}carbamate

To a solution of Intermediate 14 (0.35 g, 0.66 mmol) in EtOAc (10 mL) were added 3-(2,2,2-trifluoroethyl) imidazole-4-carboxylic acid (Journal of Fluorine Chemistry, 2000, 106, 189-197) (0.26 g, 1.32 mmol), pyridine (0.27 mL, 3.29 mmol) and T₃P® (50% in EtOAc) (1.96 mL, 3.29 mmol) at r.t. The reaction mixture was heated at 60° C. for 16 h, then poured into cold water (60 mL) and extracted with EtOAc (2×60 mL). The organic layer was separated, then dried over anhydrous Na₂SO₄ and concentrated in vacuo. The crude residue was purified by column chromatography (silica, 100-200 mesh, 40% EtOAc in hexanes). The resulting material was subjected to chiral preparative HPLC (Column: Phenomenex Cellulose-4, 250 mm×4.6 mm, 5 μm; Mobile Phase A: n-hexane+0.1% isopropylamine; Mobile Phase B: EtOH/MeOH (50:50); Flow rate: 1.0 mL/minute; isocratic: 20% B) to afford the title compound (0.095 g, 21%) as an off-white solid. δ_{H} (400 MHz, DMSO-d₆) 10.53 (s, 1H), 10.12 (s, 1H), 8.04 (s, 1H), 7.94 (s, 1H), 7.47-7.55 (m, 1H), 7.39-7.47 (m, 1H), 7.24-7.34 (m, 1H), 5.33-5.43 (m, 2H), 4.47-4.63 (m, 1H), 3.63 (d, J 16.14 Hz, 1H), 3.43-3.53 (m, 1H), 3.27 (s, 3H), 2.18-2.43 (m, 2H), 1.91-2.08 (m, 1H), 1.79 (s, 3H), 1.60-1.75 (m, 1H), 1.45 (s, 9H), 1.20-1.38 (m, 2H), 0.86 (t, J 6.36 Hz, 1H). Chiral HPLC: RT 11.72 minutes (RT for opposite diastereomer: 13.84 minutes).

### EXAMPLE 1

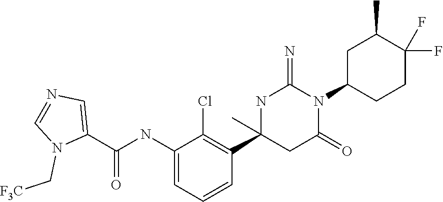 [SMILES: C[C@@H]1C[C@H](N2C(=N)N[C@](C)(c3cccc(NC(=O)c4cncn4CC(F)(F)F)c3Cl)CC2=O)CCC1(F)F]

### N-(2-Chloro-3-{(4S)-1-[(1R,3R)-4,4-difluoro-3-methylcyclohexyl]-2-imino-4-methyl-6-oxohexahydropyrimidin-4-yl}phenyl)-3-(2,2,2-trifluoroethyl)imidazole-4-carboxamide

To a solution of Intermediate 20 (0.085 g, 0.12 mmol) in DCM (8 mL) was added TFA (0.19 mL, 2.42 mmol) at 0° C. The reaction mixture was stirred at r.t. for 3 h, then concentrated in vacuo. The residue was diluted with saturated aqueous NaHCO₃ solution (30 mL) and water (50 mL), and extracted with DCM (2×40 mL). The organic layer was separated, washed with water (50 mL) and brine (50 mL), then dried over anhydrous Na₂SO₄ and concentrated in vacuo. The crude residue was purified by preparative HPLC to afford the title compound (0.038 g, 54%) as an off-white solid. δ_{H} (400 MHz, DMSO-d₆) 0.83 (d, J 6.36 Hz, 3H), 0.99-1.19 (m, 2H), 1.47-1.68 (m, 3H), 1.69-1.92 (m, 2H), 1.94-2.08 (m, 2H), 2.74-2.91 (m, 1H), 3.49-3.66 (m, 1H), 3.80-3.99 (m, 1H), 4.61-4.82 (m, 1H), 5.31-5.48 (m, 2H), 5.92-6.14 (m, 2H), 7.32-7.46 (m, 3H), 7.93 (s, 1H), 8.03 (s, 1H), 10.05 (s, 1H). LCMS (Method 1, ESI): 561.20 [MH]⁺, RT 1.84 minutes.

### Preparation of Hydrochloride Salt

To a solution of Example 1 (0.035 g, 0.06 mmol) in DCM (8 mL) was added 4M HCl in 1,4-dioxane (0.03 mL, 0.12 mmol) at 0° C. The reaction mixture was stirred at r.t. for 20 minutes, then concentrated in vacuo. The crude residue was washed with diethyl ether (2 mL) and pentane (5 mL), then dried in vacuo, to afford Example 1 (hydrochloride salt) (0.03 g, 82%) as an off-white solid. δ_{H} (400 MHz, DMSO-d₆) 0.77 (d, J 2.93 Hz, 3H), 0.82-0.93 (m, 2H), 1.77 (s, 3H), 1.82-2.07 (m, 5H), 2.22-2.37 (m, 1H), 3.24-3.37 (m, 1H), 3.86 (s, 1H), 5.29-5.50 (m, 2H), 7.23-7.37 (m, 1H), 7.38-7.48 (m, 1H), 7.48-7.58 (m, 1H), 7.94-8.36 (m, 2H), 8.66-9.22 (m, 2H), 10.21 (m, 1H), 10.62 (s, 1H). LCMS (Method 2, ESI): 561.20 [MH]⁺, RT 2.44 minutes.

### EXAMPLE 2

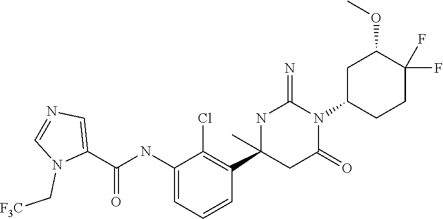 [SMILES: CO[C@H]1C[C@@H](N2C(=N)N[C@](C)(c3cccc(NC(=O)c4cncn4CC(F)(F)F)c3Cl)CC2=O)CCC1(F)F]

### N-(2-Chloro-3-{(4S)-1-[(1S*, 3S*)-4,4-difluoro-3-methoxycyclohexyl]-2-imino-4-methyl-6-oxohexahydropyrimidin-4-yl}phenyl)-3-(2,2,2-trifluoroethyl)imidazole-4-carboxamide hydrochloride

To a solution of Intermediate 21 (0.09 g, 0.13 mmol) in 1,4-dioxane (2 mL) was added 4M HCl in 1,4-dioxane (1.66 mL, 6.63 mmol) at 0° C. The reaction mixture was stirred at r.t. for 5 h, then concentrated in vacuo. The crude residue was washed with diethyl ether (4 mL) and n-pentane (6 mL), then dried in vacuo, to afford the title compound (0.067 g, 81%) as an off-white solid. δ_{H} (400 MHz, DMSO-d₆) 10.72 (s, 1H), 10.26 (s, 1H), 9.06 (br s, 2H), 8.26 (s, 1H), 8.05 (s, 1H), 7.51-7.58 (m, 1H), 7.40-7.50 (m, 1H), 7.35 (d, J 7.82 Hz, 1H), 5.37-5.51 (m, 2H), 3.66-3.76 (m, 1H), 3.39-3.49 (m, 2H), 3.33 (d, J 16.14 Hz, 1H), 3.24 (s, 3H), 2.19-2.38 (m, 1H), 1.93-2.08 (m, 2H), 1.81-1.92 (m, 1H), 1.78 (s, 3H), 1.15-1.27 (m, 1H). One proton signal merged into solvent peak. LCMS (Method 2, ESI): 577.00 [MH]⁺, RT 2.05 minutes.

1. A compound of formula (I) or a pharmaceutically acceptable salt thereof: 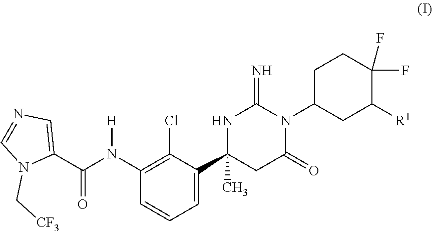
wherein
R¹ represents methyl or methoxy.

2. A compound as claimed in claim 1 represented by formula (IA): 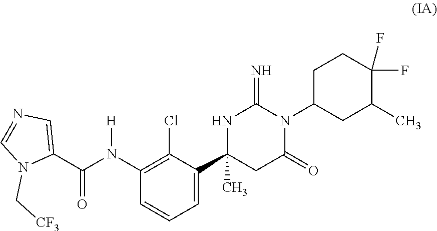 [SMILES: CC1CC(N2C(=N)N[C@](C)(c3cccc(NC(=O)c4cncn4CC(F)(F)F)c3Cl)CC2=O)CCC1(F)F]
or a pharmaceutically acceptable salt thereof.

3. A compound as claimed in claim 1 represented by formula (IB): 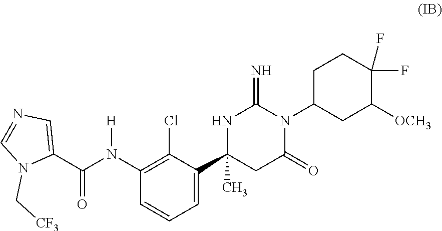 [SMILES: COC1CC(N2C(=N)N[C@](C)(c3cccc(NC(=O)c4cncn4CC(F)(F)F)c3Cl)CC2=O)CCC1(F)F]
or a pharmaceutically acceptable salt thereof.

4. A compound as claimed in any one of the preceding claims wherein the 3-substituted 4,4-difluorocyclohex-1-yl moiety present therein is in the 1R,3R or 1S,3S configuration.

5. A compound as claimed in claim 4 wherein the 3-substituted 4,4-difluoro-cyclohex-1-yl moiety is in the 1R,3R configuration.

6. A compound as claimed in claim 4 wherein the 3-substituted 4,4-difluoro-cyclohex-1-yl moiety is in the 1S,3S configuration.

7. N-(2-Chloro-3-{(4S)-1-[(1R,3R)-4,4-difluoro-3-methylcyclohexyl]-2-imino-4-methyl-6-oxohexahydropyrimidin-4-yl}phenyl)-3-(2,2,2-trifluoroethyl)imidazole-4-carboxamide.

8. The hydrochloride salt of N-(2-chloro-3-{(4S)-1-[(1R,3R)-4,4-difluoro-3-methylcyclohexyl]-2-imino-4-methyl-6-oxohexahydropyrimidin-4-yl}phenyl)-3-(2,2,2-trifluoroethyl) imidazole-4-carboxamide.

9. N-(2-Chloro-3-{(4S)-1-[(1S*,3S*)-4,4-difluoro-3-methoxycyclohexyl]-2-imino-4-methyl-6-oxohexahydropyrimidin-4-yl}phenyl)-3-(2,2,2-trifluoroethyl)-imidazole-4-carboxamide.

10. The hydrochloride salt of N-(2-chloro-3-{(4S)-1-[(1S*,3S*)-4,4-difluoro-3-methoxycyclohexyl]-2-imino-4-methyl-6-oxohexahydropyrimidin-4-yl}phenyl)-3-(2,2,2-trifluoroethyl) imidazole-4-carboxamide.

11. A compound of formula (I) as defined in claim 1, or a pharmaceutically acceptable salt thereof, for use in therapy.

12. A compound of formula (I) as defined in claim 1, or a pharmaceutically acceptable salt thereof, for use in the treatment and/or prevention of malaria.

13. A pharmaceutical composition comprising a compound of formula (I) as defined in claim 1, or a pharmaceutically acceptable salt thereof, in association with a pharmaceutically acceptable carrier.

14. The use of a compound of formula (I) as defined in claim 1, or a pharmaceutically acceptable salt thereof, for the manufacture of a medicament for the treatment and/or prevention of malaria.

15. A method for the treatment and/or prevention of malaria, which comprises administering to a patient in need of such treatment an effective amount of a compound of formula (I) as defined in claim 1, or a pharmaceutically acceptable salt thereof.
